SEQUENCE LISTING (5 sequences)

SEQ ID NO: 1 - DNA, 15 bp, Homo sapiens
caaactagaatacat

SEQ ID NO: 2 - DNA, 15 bp, Homo sapiens
tcgttgggggaaggc

SEQ ID NO: 3 - DNA, 15 bp, Homo sapiens
ccaactgtgatgcat

SEQ ID NO: 4 - DNA, 15 bp, Homo sapiens
caaactgggatacat

SEQ ID NO: 5 - DNA, 15 bp, Homo sapiens
tcgactgtgatgcat

(19) United States
(12) Patent Application Publication
(10) Pub. No.: US 2009/0209622 A1
(43) Pub. Date: Aug. 20, 2009

(54) Diagnosis of anxiety disorders

(76) Inventors: Jordan W. Smoller, Newton, MA (US); Martin P. Paulus, La Jolla, CA (US); Joel Gelernter, New Haven, CT (US); Murray B. Stein, La Jolla, CA (US)

Correspondence Address: WOLF GREENFIELD & SACKS, P.C., 600 ATLANTIC AVENUE, BOSTON, MA, 02210-2206, US

(21) Appl. No.: 11/986,553

(22) Filed: Nov. 21, 2007

Related U.S. Application Data
(60) Provisional application No. 60/860,297, filed on Nov. 21, 2006.

(51) Int. Cl. C12Q 1/68; C07H 21/04; A61K 31/7088; A61P 25/24

(52) U.S. Cl. 514/44 A; 435/6; 536/24.3

(57) ABSTRACT

The invention relates to methods for diagnosing a genetic predisposition or susceptibility for anxiety disorders in a human. The methods include detecting particular markers at the human RGS2 locus in a sample obtained from the human. The invention also relates to the improved diagnosis that is based on the analysis of haplotypes for the RGS2 locus.

### RELATED APPLICATION

This application claims the benefit under 35 U.S.C. §119(e) of U.S. provisional application 60/860,297, filed Nov. 21, 2006, the entire contents of which are incorporated by reference herein.

### GOVERNMENT SUPPORT

This work was funded in part by the National Institutes of Health under NIMH grants K-08 MH01770, R01MH47077-11, K24 MH64122 and R01 MH65413, and NIDA grants DA12690, DA12849, and DA15105. The government has certain rights in this invention.

### FIELD OF THE INVENTION

The invention relates to methods for diagnosing a genetic predisposition or susceptibility for anxiety disorders in a human. The methods include detecting particular markers at the human RGS2 locus in a sample obtained from the human. The invention also relates to the improved diagnosis that is based on the analysis of haplotypes for the RGS2 locus.

### BACKGROUND OF THE INVENTION

Anxiety disorders are the most common class of psychiatric disorders and are associated with a substantial burden of illness and more than $80 billion in annual costs to society. Anxiety disorders are known to be familial and heritable¹, but the identification of susceptibility genes has been difficult to accomplish for several reasons. The etiology of these disorders is thought to reflect the effects of multiple genes of individually modest effect interacting with environmental factors. Limited understanding of the underlying neurobiology means that many genes are plausible risk candidates, but few are compelling. Beyond this genetic and biological complexity, there is considerable phenotypic complexity for anxiety disorders. While the constellations of symptoms used as diagnostic criteria in the DSM-IV have been useful for clinical practice, it is unlikely that they are the optimal phenotype definitions for genetic analyses¹. Indeed, family, twin and linkage studies suggest that genes confer susceptibility to anxiety proneness in a manner that cuts across clinical diagnostic labels¹⁻³. Although identifying anxiety susceptibility genes is a formidable challenge, the existence of well-validated animal models and intermediate phenotypes, including anxious temperament and functional neuroimaging phenotypes, provide crucial tools.

### SUMMARY OF THE INVENTION

The gene encoding regulator of G protein signaling 2 (Rgs2) has been identified as a quantitative trait locus influencing anxiety behavior in mice. Here we report that markers at the human RGS2 locus are associated with (1) childhood behavioral inhibition (a temperamental precursor of social anxiety disorder), (2) introversion (a core personality trait for social anxiety disorder), and (3) increased bilateral insular cortex activation during emotion processing (the extent of which correlates with measures of introversion and social interactional anxiety) in two independent samples of young adults. Taken together, these results provide the first evidence that a gene influencing anxiety in mice is also associated with intermediate phenotypes for human anxiety disorders across multiple levels of assessment, including temperament, personality, and brain function. This translational research suggests that pharmacological modulation of RGS2 function may provide a novel therapeutic approach to the treatment of anxiety disorders.

According to one aspect of the invention, methods for diagnosing an anxiety disorder in a human individual are provided. In particular, the methods permit diagnosis of a genetic predisposition or susceptibility for an anxiety disorder. The methods include detecting in a sample obtained from the individual one or more genetic markers in a RGS2 nucleic acid or fragment thereof. Preferably the one or more genetic markers are rs3856223, rs6670601, rs6670801, rs10801152, rs10921267, rs6428136, rs7531013, rs1342809, rs1890397, rs2746071, rs2746073, rs17647363, rs4606, rs3767488 and/or rs1819741. The presence of the one or more markers indicates that the individual has a genetic predisposition or susceptibility for an anxiety disorder. In preferred embodiments, the one or more markers are rs10801152, rs10921267, rs6428136, rs1342809, rs2746071, rs2746073, rs4606, rs3767488 and/or rs1819741; more preferably the markers are rs10801152, rs4606 and/or rs1819741, and more preferably still the one or more markers is rs4606.

In other embodiments, the method comprises detecting a haplotype. In preferred embodiments, the haplotype includes the markers rs10801152, rs4606, rs6428136 and rs1819741, or includes all 15 markers or the markers described above.

In certain embodiments, the anxiety disorder is inhibited temperament (BI), introversion, panic anxiety disorder, phobic anxiety disorder, or social anxiety disorder (SAD). In preferred embodiments, the anxiety disorder is social phobia.

In still other embodiments, the nucleic acid is DNA, genomic DNA, RNA, cDNA, hnRNA or mRNA. In some embodiments, detection is accomplished by sequencing, hybridization, restriction fragment analysis, oligonucleotide ligation assay or allele specific PCR.

According to another aspect of the invention, diagnostic kits and/or research kits are provided. The kits include at least one combination of probes for detecting at least one of the markers described above.

Determination of the presence or absence of the markers at the RGS2 locus described herein may guide therapy. Thus, according to another aspect of the invention, methods of treatment or prophylaxis of an anxiety disorder are provided. The methods include performing a diagnostic method described herein to identify an individual that has a genetic predisposition or susceptibility for an anxiety disorder, and administering to the individual a therapeutically effective amount of a composition suitable to delay, reduce or prevent the anxiety disorder in the individual and/or treating the individual with therapy.

In some embodiments, the composition comprises an antidepressant compound. Preferred antidepressant compounds include monoamine oxidase inhibitors (MAOI) such as Harmaline, Iproclozide, Iproniazid, Isocarboxazid, Nialamide, Phenelzine, Selegiline, Toloxatone, Tranylcypromine; reversible inhibitors of monoamine oxidase A (RIMA) such as Brofaromine and Moclobemide; dopamine reuptake inhibitors (DARI) such as Amineptine, Phenmetrazine, Vanoxerine and Modafinil; norepinephrine-dopamine reuptake inhibitors such as Bupropion; norepinephrine reuptake inhibitors (NRI) or (NARI) such as Atomoxetine, Maprotiline, Reboxetine and Viloxazine; serotonin-norepinephrine reuptake inhibitors (SNRI) such as Duloxetine, Milnacipran and Venlafaxine; selective serotonin reuptake inhibitors (SSRI) such as Alaproclate, Etoperidone, Citalopram, Escitalopram, Fluoxetine, Fluvoxamine, Paroxetine, Sertraline and Zimelidine; selective serotonin reuptake enhancers (SSRE) such as Tianeptine; tricyclic antidepressants (TCA) such as Amitriptyline, Amoxapine, Butriptyline, Clomipramine, Desipramine, Dibenzepin, Dothiepin, Doxepin, Imipramine, Iprindole, Lofepramine, Melitracen, Nortriptyline, Opipramol, Protriptyline and Trimipramine; tetracyclic antidepressants such as Maprotiline, Mianserin, Nefazodone and Trazodone; or noradrenergic and specific serotonergic antidepressants (NaSSA) such as Mirtazapine.

In other embodiments, the composition includes a RGS2 modulator, such as a siRNA molecule that reduces RGS2 expression, or an expression vector that increases expression of RGS2, thereby increasing RGS2 activity.

In still other embodiments, the composition comprises an antidepressant and a RGS2 modulator, which may be administered together or separately.

In further embodiments, the therapy is psychiatric therapy, psychotherapy, cognitive-behavioral therapy and/or behavior therapy. Combinations of such therapies and pharmaceuticals may be used in treatment.

The diagnostic methods of the invention also can be used to determine when not to treat an individual suspected of having an anxiety disorder for the anxiety disorder.

These and other aspects of the invention, as well as various embodiments thereof, will become more apparent in reference to the drawings and detailed description of the invention.

### BRIEF DESCRIPTION OF THE DRAWINGS

FIG. 1. Schematic of RGS2 locus, depicting positions of genotyped SNPs. The lower portion of the figure shows a magnification of the RGS2 locus.

FIG. 2. Linkage disequilibrium and association analyses for RGS2 in the BI family sample. Gene position for the RGS2 locus is shown above the chromosome bar. Relative positions of genotyped SNP markers are shown and significant single marker association results are depicted with colored circles. Haplotypes with frequencies >5% are shown below the markers and the associated haplotype is shaded in green. Pairwise marker linkage disequilibrium using the D′ statistic is indicated by the matrix at bottom (red=strong LD; white=weak LD). Figure drawn using Locusview (T. Petryshen and A. Kirby, unpublished software; http://www.broad.mit.edu/mpg/locusview)

FIG. 3. Activation differences associated with the rs4606 genotype in left amygdala for the combined 1.5 T and 3 T samples. The marginal means are obtained relative to the covaried ancestral proportion weights and field strength indicators. Active voxels are volume-thresholded at an a-posteriori p<0.05; associated bar graphs show the % signal difference between face emotion processing and the sensorimotor control condition (adjusted for ancestral proportion weights and field strength indicators).

FIG. 4. Activation differences associated with the rs4606 genotype in bilateral insula for the combined 1.5 T and 3 T samples. The marginal means are obtained relative to the covaried ancestral proportion weights and field strength indicators. For the statistical effect see supplemental materials. Axial slices in Talairach coordinates at z=0 and z=4 show two regions on the left and one on the right, the bar graphs show the larger of the two areas within the left insula.

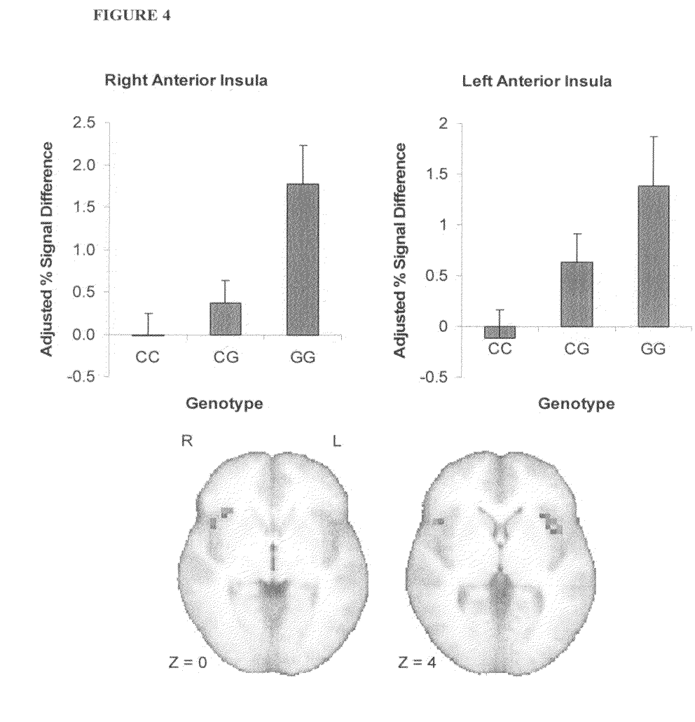

FIG. 5. rs10801152 genotype effect —marginal signal difference (face-shape) as a function of genotype adjusted for magnet strength and ancestral proportion.

FIG. 6. This figure shows the results of ANCOVA with magnet and rs4606 genotype as the main variables and AIM1 and AIM2 as covariates. All values for the bar graphs are corrected for the covariates. As shown, the risk allele rs4606-G was associated with increased activation during the faces relative to the sensorimotor control condition in all areas identified in the pooled magnet analyses described in the main text.

### DETAILED DESCRIPTION OF THE INVENTION

Here we report evidence that genetic variants at the RGS2 locus are associated with human social anxiety related phenotypes including behavioral measures of childhood inhibited temperament (BI), personality measures of social anxiety (introversion), and neuroimaging measures of emotion processing previously linked to social anxiety in adults.

A quantitative trait locus (QTL) on mouse chromosome 1 has been the most widely replicated locus linked to anxious temperament phenotypes in mice⁴. Yalcin and colleagues⁵ fine-mapped this locus and identified the gene encoding Regulator of G-protein signaling 2 (Rgs2) as a quantitative trait gene underlying this linkage signal. Rgs2 knockout mice exhibit increased anxiety and fear behavior, altered hippocampal synaptic plasticity, and elevated sympathetic tone. The Rgs2 protein, which is expressed in cortical and limbic brain regions, is part of a family of proteins that accelerate deactivation of G proteins to reduce G protein coupled receptor (GPCR) signaling⁶. Neurotransmitters strongly implicated in the biology of anxiety, including serotonin and norepinephrine, act at GPCRs. We previously observed modest evidence of linkage between markers encompassing RGS2 and a phenotype of anxiety disorder proneness in a targeted genome screen³. Thus, convergent evidence implicates RGS2 as compelling candidate locus underlying anxiety proneness in humans. We examined whether variation at the RGS2 locus influences intermediate phenotypes for anxiety disorder at the level of behavior and brain function. We first examined association of RGS2 markers with behavioral inhibition to the unfamiliar (BI), an anxiety-related form of temperament characterized by a tendency to be shy, avoidant, and behaviorally-restrained in situations that are novel or unfamiliar⁷. Mouse models of unconditioned and novelty-induced fear responses closely parallel behavioral and biological features of human BI, including inhibition of behavior and increased sympathetic nervous system reactivity⁸. We next examined the BI-related adult personality trait of introversion (low extraversion), which is also characterized by inhibition and avoidant behavior and, finally, limbic responses to emotional faces on fMRI, a neuroimaging phenotype linked to BI⁹, anxiety proneness¹⁰, and social anxiety disorder^{11,12}.

The term “allele” is used herein to refer to variants of a nucleotide sequence. A biallelic polymorphism has two forms. Typically the first identified allele is designated as the original allele whereas other alleles are designated as alternative alleles. Diploid organisms is homozygous or heterozygous for an allelic form.

The term “genotype” as used herein refers the identity of the alleles present in an individual or a sample. The term “genotyping” a sample or an individual for an allelic marker consists of determining the specific allele or the specific nucleotide carried by an individual at an allelic marker.

The term “haplotype” refers to a combination of alleles present in an individual or a sample.

The methods described herein use RGS2 SNP markers. As used herein, the term “SNP” includes all single base variants and also includes single nucleotide insertions and deletions in addition to single nucleotide substitutions (e.g., A->G). A single nucleotide polymorphism occurs at a polymorphic site occupied by a single nucleotide, which is the site of variation between allelic sequences. The typical frequency at which SNPs are observed is about 1 per 1000 base pairs (Li and Sadler, Genetics, 129:513-523, 1991; Wang et al., Science, 280:1077-1082, 1998; Harding et al., Am. J. Human Genet., 60:772-789, 1997; Taillon-Miller et al., Genome Res., 8:748-754, 1998).

Typically, between different genomes or between different individuals, the polymorphic site is occupied by two different nucleotides. SNPs occur at defined positions within genomes and can be used for gene mapping, defining population structure, and performing functional studies. SNPs are useful as markers because many known genetic diseases are caused by point mutations and insertions/deletions. The conformation of the nucleic acid molecule is generally detectable, identifiable and/or distinguishable using methods known in the art, such as electrophoretic mobility as measured by gel electrophoresis, capillary electrophoresis, and/or susceptibility to endonuclease digestion etc.

“Linkage” describes the tendency of genes, alleles, loci or genetic markers to be inherited together as a result of their location on the same chromosome, and can be measured by percent recombination between the two genes, alleles, loci or genetic markers. Loci occurring within 50 centimorgan of each other are linked. Some linked markers occur within the same gene or gene cluster.

“Linkage disequilibrium” or “allelic association” means the preferential association of a particular allele or genetic marker with a specific allele, or genetic marker at a nearby chromosomal location more frequently than expected by chance for any particular allele frequency in the population. Linkage disequilibrium may result from natural selection of certain combination of alleles or because an allele has been introduced into a population too recently/to have reached equilibrium with linked alleles.

A marker in linkage disequilibrium can be particularly useful in detecting susceptibility to disease (or other phenotype) notwithstanding that the marker does not cause the disease. For example, a marker (X) that is not itself a causative element of a disease, but which is in linkage disequilibrium with a gene (including regulatory sequences) (Y) that is a causative element of a phenotype, can be used detected to indicate susceptibility to the disease in circumstances in which the gene Y may not have been identified or may not be readily detectable. Younger alleles (i.e., those arising from mutation relatively late in evolution) are expected to have a larger genomic sequencement in linkage disequilibrium. The age of an allele can be determined from whether the allele is shared between ethnic human groups and/or between humans and related species.

“Genetic variant” or “variant” means a specific genetic variant which is present at a particular genetic locus in at least one individual in a population and that differs from a reference sequence.

By “therapeutically effective amount” is meant an amount of an agent which relieves to some extent one or more symptoms of the disease or disorder in the patient; or returns to normal either partially or completely one or more physiological or biochemical parameters associated with or causative of the disease.

In a particular embodiment, the individual is an individual at risk for development of an anxiety disorder. In another embodiment the individual exhibits clinical symptomology associated with an anxiety disorder. In one embodiment, the individual has been clinically diagnosed as having an anxiety disorder. In a preferred embodiment, the anxiety disorder is social phobia. Anxiety disorders can be evaluated as known in the art. Human social anxiety related phenotypes including behavioral measures of childhood inhibited temperament (BI), personality measures of social anxiety, e.g., introversion (low extraversion) which is also characteristic of individuals with panic and phobic anxiety disorders, including social anxiety disorder, and neuroimaging measures of emotion processing previously linked to social anxiety in adults.

The genetic material to be assessed can be obtained from any nucleated cell from the individual. The nucleic acids used in the methods according to the present invention can be DNA, genomic DNA, RNA, cDNA, hnRNA and/or mRNA. For assay of genomic DNA, virtually any biological sample (other than pure red blood cells) is suitable. For example, convenient tissue samples include whole blood, semen, saliva, tears, urine, fecal material, sweat, skin and hair. For assay of cDNA or mRNA, the tissue sample must be obtained from an organ in which the target nucleic acid is expressed. For example, cells from the central nervous system (such as cells of the hippocampus), heart, brain, lung and skeletal muscle are suitable sources for obtaining cDNA for the RGS2 gene.

Applicable diagnostic techniques include, but are not limited to, DNA sequencing including mini-sequencing, primer extension, hybridization with allele-specific oligonucleotides, oligonucleotide ligation assays, PCR using allele-specific primers, dot blot analysis, flap probe cleavage approaches, restriction fragment length polymorphism, kinetic PCR, and PCR-SSCP, fluorescent in situ hybridisation, pulsed field gel electrophoresis analysis, Southern blot analysis, single stranded conformation analysis, denaturing gradient gel electrophoresis, temperature gradient gel electrophoresis, denaturing HPLC and RNAse protection assays, all of which are presently known to the person skilled in the art and routinely practiced in the art.

Many of the methods described herein require amplification of DNA from target samples. This can be accomplished by e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The nucleotides which occupy the polymorphic sites of interest can be identified by a variety methods, such as Southern analysis of genomic DNA; direct mutation analysis by restriction enzyme digestion; Northern analysis of RNA; denaturing high pressure liquid chromatography (DHPLC); gene isolation and sequencing; hybridization of an allele-specific oligonucleotide with amplified gene products; single base extension (SBE); A sampling of suitable procedures are discussed below.

The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-50° C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

Some probes are designed to hybridize to a segment of target DNA such that the polymorphic site aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

The polymorphisms can also be identified by hybridization to nucleic acid arrays (e.g., microarrays), some examples of which are described in WO 95/11995. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic site and the other of which exhibits perfect complementarity to a distal site. The single base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

The direct analysis of the sequence of polymorphisms of the present invention can be accomplished using any known nucleic acid sequencing method, including the dideoxy chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W. H. Freeman and Co, New York, 1992), Chapter 7.

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., (PNAS 94:10756-61 (1997), incorporated herein by reference) uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently labeled dideoxyribonucleotides (ddNTPs) in dye-terminator sequencing fashion, except that no deoxyribonucleotides are present. An increase in fluorescence of the added ddNTP in response to excitation at the wavelength of the labeled primer is used to infer the identity of the added nucleotide.

More than one phenotypic trait may be affected by the SNPs described herein. For example, other neuropsychiatric disorders which are believed to be alternate expressions of an anxiety disorder may also be affected by the RGS2 polymorphisms described herein. Additionally, the described polymorphisms may predispose an individual to a distinct mutation that is causally related to a certain phenotype, such as susceptibility or resistance to anxiety disorder. The discovery of these polymorphisms and their correlation with anxiety disorder facilitates biochemical analysis of the variants and the development of assays to characterize the variants and to screen for pharmaceuticals that interact directly with one or another form of the protein.

Alternatively, a polymorphism may be one of a group of two or more polymorphisms in the RGS2 gene, or in linkage disequilibrium with such polymorphisms, that form a haplotype which contributes to the presence, absence or severity of the anxiety disorder, e.g., social phobia. An assessment of other polymorphisms within the RGS2 gene, or in linkage disequilibrium with such polymorphisms, can be undertaken, and the separate and combined effects of these polymorphisms on the anxiety disorder phenotype can be assessed.

Correlation between a particular phenotype, e.g., the anxiety phenotype, and the presence or absence of a particular allele is performed for a population of individuals who have been tested for the presence or absence of the phenotype. Correlation can be performed by standard statistical methods as known in the art and as described herein and statistically significant correlations between polymorphic form(s) and phenotypic characteristics are noted.

This correlation can be exploited in several ways. In the case of a strong correlation, detection of the polymorphic form in an individual may justify immediate administration of treatment, or at least the institution of regular monitoring of the individual. The individual can be motivated to begin simple life-style changes (e.g., therapy or counseling) that can be accomplished at little cost to the individual but confer potential benefits in reducing the risk of conditions to which the individual may have increased susceptibility by virtue of the particular allele. Furthermore, identification of a polymorphic form correlated with enhanced receptiveness to one of several treatment regimes for a disorder indicates that this treatment regime should be followed for the individual in question.

Furthermore, it may be possible to identify a physical linkage between a genetic locus associated with a trait of interest (e.g., anxiety disorder) and polymorphic markers that are not associated with the trait, but are in physical proximity with the genetic locus responsible for the trait and co-segregate with it. Such analysis is useful for mapping a genetic locus associated with a phenotypic trait to a chromosomal position, and thereby cloning gene(s) responsible for the trait. See Lander et al., Proc. Natl. Acad. Sci. (USA) 83, 7353-7357 (1986); Lander et al., Proc. Natl. Acad. Sci. (USA) 84, 2363-2367 (1987); Donis-Keller et al., Cell 51, 319-337 (1987); Lander et al., Genetics 121, 185-199 (1989)). Genes localized by linkage can be cloned by a process known as directional cloning. See Wainwright, Med. J. Australia 159, 170-174 (1993); Collins, Nature Genetics 1, 3-6 (1992).

Linkage studies are typically performed on members of a family. Available members of the family are characterized for the presence or absence of a phenotypic trait and for a set of polymorphic markers. The distribution of polymorphic markers in an informative meiosis is then analyzed to determine which polymorphic markers cosegregate with a phenotypic trait. See, e.g., Kerem et al., Science 245, 1073-1080 (1989); Monaco et al., Nature 316, 842 (1985); Yamoka et al., Neurology 40, 222-226 (1990); Rossiter et al., FASEB Journal 5, 21-27 (1991).

Linkage is analyzed by calculation of LOD (log of the odds) values. A lod value is the relative likelihood of obtaining observed segregation data for a marker and a genetic locus when the two are located at a recombination fraction theta (θ), versus the situation in which the two are not linked, and thus segregating independently (Thompson & Thompson, Genetics in Medicine (5th ed, W. B. Saunders Company, Philadelphia, 1991); Strachan, “Mapping the human genome” in The Human Genome (BIOS Scientific Publishers Ltd, Oxford), Chapter 4). A series of likelihood ratios are calculated at various recombination fractions (theta, θ), ranging from θ=0.0 (coincident loci) to θ=0.50 (unlinked). Thus, the likelihood at a given value of θ is: probability of data if loci linked at θ to probability of data if loci unlinked. The computed likelihoods are usually expressed as the log₁₀ of this ratio (i.e., a lod score). For example, a lod score of 3 indicates 1000:1 odds against an apparent observed linkage being a coincidence. The use of logarithms allows data collected from different families to be combined by simple addition. Computer programs are available for the calculation of lod scores for differing values of θ (e.g., LIPED, MLINK (Lathrop, Proc. Nat. Acad. Sci. (USA) 81, 3443-3446 (1984)). For any particular lod score, a recombination fraction may be determined from mathematical tables. See Smith et al., Mathematical tables for research workers in human genetics (Churchill, London, 1961); Smith, Ann. Hum. Genet. 32, 127-150 (1968). The value of θ at which the lod score is the highest is considered to be the best estimate of the recombination fraction.

Positive lod score values suggest that the two loci are linked, whereas negative values suggest that linkage is less likely (at that value of .theta.) than the possibility that the two loci are unlinked. By convention, a combined lod score of +3 or greater (equivalent to greater than 1000:1 odds in favor of linkage) is considered definitive evidence that two loci are linked. Similarly, by convention, a negative lod score of −2 or less is taken as definitive evidence against linkage of the two loci being compared. Negative linkage data are useful in excluding a chromosome or a segment thereof from consideration. The search focuses on the remaining non-excluded chromosomal locations.

Another aspect of the present invention is directed to methods of treatment or prophylaxis in a mammal, in particular a human, that has a genetic predisposition or susceptibility for an anxiety disorder, e.g., social phobia. The methods includes performing the diagnostic method according to the present invention, and based on the results of the diagnostic method, administering to the mammal an effective dose of a therapeutic composition suitable to delay, reduce or prevent the anxiety disorder in the mammal. Preferably, the therapeutic composition comprises an antidepressant compound as are known in the art, such as monoamine oxidase inhibitors (MAOI) including Harmaline, Iproclozide, Iproniazid, Isocarboxazid, Nialamide, Phenelzine (e.g., Nardil), Selegiline (e.g., Emsam/Eldepryl), Toloxatone, Tranylcypromine (e.g., Parnate); reversible inhibitors of monoamine oxidase A (RIMA) including Brofaromine and Moclobemide; dopamine reuptake inhibitors (DARI) including Amineptine, Phenmetrazine, Vanoxerine and Modafinil; norepinephrine-dopamine reuptake inhibitors including Bupropion (e.g., Wellbutrin, Zyban); norepinephrine reuptake inhibitors (NRI) or (NARI) including Atomoxetine, Maprotiline, Reboxetine and Viloxazine; serotonin-norepinephrine reuptake inhibitors (SNRI) including Duloxetine (e.g., Cymbalta), Milnacipran and Venlafaxine (e.g., Effexor, Efexor); selective serotonin reuptake inhibitors (SSRI) including Alaproclate, Etoperidone, Citalopram (e.g., Celexa, Cipramil, Talohexane), Escitalopram (e.g., Lexapro, Cipralex), Fluoxetine (e.g., Prozac, Sarafem, Fluctin, Fontex, Prodep, Fludep, Lovan), Fluvoxamine (e.g., Luvox, Faverin), Paroxetine (e.g., Paxil, Seroxat, Aropax), Sertraline (e.g., Zoloft, Lustral, Apo-Sertral, Asentra, Gladem, Serlift, Stimuloton) and Zimelidine; selective serotonin reuptake enhancers (SSRE) including Tianeptine; tricyclic antidepressants (TCA) including Amitriptyline (e.g., Elavil), Amoxapine, Butriptyline, Clomipramine, Desipramine, Dibenzepin, Dothiepin (e.g., Prothiaden, Dothapax), Doxepin, Imipramine, Iprindole, Lofepramine, Melitracen, Nortriptyline, Opipramol, Protriptyline and Trimipramine; tetracyclic antidepressants including Maprotiline, Mianserin, Nefazodone and Trazodone; and noradrenergic and specific serotonergic antidepressants (NaSSA) including Mirtazapine (e.g., Remeron). Antianxiety drugs, such as Chlordiazepoxide (Librium) and diazepam (Valium) also may be used for treatment alone or in combination with antidepressants.

Individuals diagnosed as predisposed to anxiety disorders may also be treated with compounds that modulate RGS2 function or activity. The modulator treatment can be combined with the therapeutics described herein, or may be provided alone. One possible modulator is an inhibitor molecule that inhibits RGS2 function or reduces expression of RGS2, such as a siRNA or antisense molecule. In one particular embodiment, the inhibitor is an antisense oligonucleotide or siRNA molecule that selectively binds to a RGS2 nucleic acid molecule, to reduce the expression of the encoded RGS2 gene product in a cell.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

As used herein, a “siRNA molecule” is a double stranded RNA molecule (dsRNA) consisting of a sense and an antisense strand, which are complementary (Tuschl, T. et al., 1999, Genes & Dev., 13:3191-3197; Elbashir, S. M. et al., 2001, EMBO J., 20:6877-6888). In one embodiment the last nucleotide at the 3′ end of the antisense strand may be any nucleotide and is not required to be complementary to the region of the target gene. The siRNA molecule may be 19-23 nucleotides in length in some embodiments. In other embodiments, the siRNA is longer but forms a hairpin structure of 19-23 nucleotides in length. In still other embodiments, the siRNA is formed in the cell by digestion of double stranded RNA molecule that is longer than 19-23 nucleotides. The siRNA molecule preferably includes an overhang on one or both ends, preferably a 3′ overhang, and more preferably a two nucleotide 3′ overhang on the sense strand. In another preferred embodiment, the two nucleotide overhang is thymidine-thymidine (TT). The siRNA molecule corresponds to at least a portion of the RGS2 gene of interest. In a preferred embodiment the first nucleotide of the siRNA molecule is a purine. Many variations of siRNA and other double stranded RNA molecules useful for RNAi inhibition of gene expression will be known to one of ordinary skill in the art.

The siRNA molecules can be plasmid-based. In a preferred method, a polypeptide encoding sequence of the RGS2 gene is amplified using the well known technique of polymerase chain reaction (PCR). The use of the entire polypeptide encoding sequence is not necessary; as is well known in the art, a portion of the polypeptide encoding sequence is sufficient for RNA interference. For example, the PCR fragment can be inserted into a vector using routine techniques well known to those of skill in the art. The insert can be placed between two promoters oriented in opposite directions, such that two complementary RNA molecules are produced that hybridize to form the siRNA molecule. Alternatively, the siRNA molecule is synthesized as a single RNA molecule that self-hybridizes to form a siRNA duplex, preferably with a non-hybridizing sequence that forms a “loop” between the hybridizing sequences. Preferably the nucleotide encoding sequence is part of the coding sequence of the RGS2 gene. The siRNA can be expressed from a vector introduced into cells.

Vectors comprising RGS2 gene sequences are provided for production of siRNA, preferably vectors that include promoters active in mammalian cells. Non-limiting examples of vectors are the pSUPER RNAi series of vectors (Brummelkamp, T. R. et al., 2002, Science, 296:550-553; available commercially from OligoEngine, Inc., Seattle, Wash.). In one embodiment a partially self-complementary nucleotide coding sequence can be inserted into the mammalian vector using restriction sites, creating a stem-loop structure. In a preferred embodiment, the mammalian vector comprises the polymerase-III H1-RNA gene promoter. The polymerase-III H1-RNA promoter produces a RNA transcript lacking a polyadenosine tail and has a well-defined start of transcription and a termination signal consisting of five thymidines (T5) in a row. The cleavage of the transcript at the termination site occurs after the second uridine and yields a transcript resembling the ends of synthetic siRNAs containing two 3′ overhanging T or U nucleotides. Other promoters useful in siRNA vectors will be known to one of ordinary skill in the art.

Vector systems for siRNA expression in mammalian cells include pSUPER RNAi system described above. Other examples include but are not limited to pSUPER.neo, pSUPER.neo+gfp and pSUPER.puro (OligoEngine, Inc.); BLOCK-iT T7-TOPO linker, pcDNA1.2N5-GW/lacZ, pENTR/U6, pLenti6-GW/U6-laminshrna and pLenti6/BLOCK-iT-DEST (Invitrogen). These vectors and others are available from commercial suppliers.

It is preferred that the antisense oligonucleotide or siRNA molecule be constructed and arranged so as to bind selectively with the target under physiological conditions, i.e., to hybridize substantially more to the target sequence than to any other sequence in the target cell under physiological conditions. One of skill in the art can easily choose and synthesize any of a number of appropriate antisense or siRNA molecules for use in accordance with the present invention. In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnol. 14:840-844, 1996). Most preferably, the antisense oligonucleotides comprise a complementary sequence of 20-30 bases. For siRNA molecules, it is preferred that the molecules be 21-23 nucleotides in length, with a 3′ 2 nucleotide overhang, although shorter and longer molecules and molecules without overhangs are also contemplated as useful in accordance with the invention.

The antisense is targeted, preferably, to sites in which mRNA secondary structure is not expected (see, e.g., Sainio et al., Cell Mol. Neurobiol. 14(5):439-457, 1994) and at which polypeptides are not expected to bind. Other methods for selecting preferred siRNA sequences are known to those of skill in the art (e.g., the “siRNA Selection Program” of the Whitehead Institute for Biomedical Research (2003)).

In one set of embodiments, the antisense oligonucleotides or siRNA molecules of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors, including in situ.

In preferred embodiments, however, the antisense oligonucleotides or siRNA molecules of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose. The present invention, thus, contemplates pharmaceutical preparations containing modified antisense molecules that are complementary to and hybridizable with, under physiological conditions, the RGS2 gene, together with pharmaceutically acceptable carriers.

Another possible modulator is an expression vector that expresses functional RGS2 protein, by which RGS2 activity is increased. Suitable expression vectors are well known in the art, as are techniques for constructing, producing and administering recombinant expression vectors in order to express a protein, in this case RGS2.

In addition to treatment with pharmaceutical compounds as described above, an individual can be treated using psychotherapy, such as cognitive-behavioral therapy or behavior therapy, as is well known in the art. The therapy can be provided as a monotherapy or as an adjunct to the pharmaceutical therapy described herein.

The invention is also directed to a diagnostic kit and/or a research kit that comprises at least one probe for detecting the RGS2 SNPs that are markers for and indicative of anxiety disorders according to the present invention. The kit can contain other compounds such as enzymes, buffers, and/or dyes for performing the method(s) of the present invention. The kit can also include instructions for performing the SNP-analysis and/or the software for a statistical analysis as described herein.

Preferably the invention further provides kits comprising at least one allele-specific oligonucleotide as described herein. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting any one or more of the polymorphisms disclosed herein. Optional additional components of the kit include, for example, restriction enzymes, reverse-transcriptase or polymerase, the substrate nucleoside triphosphates, means used to label, and the appropriate buffers for reverse transcription, PCR, or hybridization reactions. Usually, the kit also contains instructions for carrying out the methods.

The invention further provides efficient methods of identifying pharmacological agents or lead compounds for agents and molecules that reduce RGS2 activity. Generally, the screening methods involve assaying for compounds which modulate the amount of activity of RGS2. As will be understood by one of ordinary skill in the art, the screening methods may measure the amount of activity directly, by using methods well known in the art. In addition, screening methods may be utilized that measure a secondary effect of RGS2 activity, for example cAMP production or Gqα signaling by 5HT2A receptors.

A wide variety of assays for pharmacological agents can be used in accordance with this aspect of the invention, including, labeled in vitro protein-protein binding assays, electrophoretic mobility shift assays, immunoassays, cell-based assays such as two- or three-hybrid screens, expression assays, etc. The assay mixture comprises a candidate pharmacological agent. Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection.

Candidate agents useful in accordance with the invention encompass numerous chemical classes, although typically they are organic compounds. Preferably, the candidate pharmacological agents are small organic compounds, i.e., those having a molecular weight of more than 50 yet less than about 2500, preferably less than about 1000 and, more preferably, less than about 500. Candidate agents comprise functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules as defined herein are also contemplated.

It is contemplated that cell-based assays as described herein can be performed using cell samples and/or cultured cells. Biopsy cells and tissues as well as cell lines grown in culture are useful in the methods of the invention.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours.

After incubation, the activity of RGS2 is detected by any convenient method available to the user. For cell-free binding type assays, a separation step is often used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximum signal to noise ratios, primarily to minimize background binding, as well as for ease of separation and cost.

Separation may be effected for example, by removing a bead or dipstick from a reservoir, emptying or diluting a reservoir such as a microtiter plate well, rinsing a bead, particle, chromatographic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet.

Detection may be effected in any convenient way for cell-based assays such as two- or three-hybrid screens, such as reporter gene transcription as described in the Examples below. For cell-free binding assays, at least one of the components usually comprises, or is coupled to, a detectable label. A wide variety of labels can be used, such as those that provide direct detection (e.g., radioactivity, luminescence, optical or electron density, energy transfer, etc.) or indirect detection (e.g., epitope tag such as the FLAG or myc epitopes, enzyme tag such as horseradish peroxidase, etc.).

A variety of methods may be used to detect the label, depending on the nature of the label and other assay components. For example, the label may be detected while bound to the solid substrate or subsequent to any separation from the solid substrate. Labels may be directly detected through optical or electron density, radioactive emissions, nonradiative energy transfers, etc. or indirectly detected with antibody conjugates, strepavidin-biotin conjugates, etc. A variety of methods for detecting the labels are well known in the art.

The present invention is described in further detail in the following non-limiting examples.

### EXAMPLES

### Methods

### Sample

Behavioral Inhibition Sample: Participants were recruited from a sample of families who had participated in a study of children at risk for anxiety disorders conducted at Massachusetts General Hospital (MGH). Details of the study sample and behavioral assessments are provided elsewhere¹³. Children from these families were classified as “inhibited” or “not inhibited” on the basis of a behavioral assessment conducted at age 21 months, 4 years, or 6 years. A total of 119 families that included at least one child who had undergone behavioral assessments were available at the time of these analyses. The self-reported race of all but nine families was Caucasian. The protocol was approved by the MGH Institutional Review Board. After complete description of the study, parents provided written informed consent for themselves and their children, who also provided oral or written assent.

NEO-E sample: Participants (n=744; 516 female) were recruited from among undergraduate psychology students at San Diego State University (SDSU). Participants had blood drawn for genetic studies, and completed questionnaires. Subjects gave informed, written consent to participate in this part of the study, which was approved by the Human Research Protection Programs at both SDSU and University of California San Diego (UCSD). Subjects received $25 for providing the blood sample. Power estimates indicate that the sample provides more than 85% power to detect an association at α<0.05 with a quantitative trait locus explaining as little as 1.5% of the trait variance.

### Neuroimaging Sample:

Participants: Initially, approximately 3000 undergraduate SDSU students participated in screening for a behavioral experiment in return for course credits. Of those individuals who participated in the behavioral study, approximately 1 in 3 expressed a willingness to participate in an fMRI study; an estimated 1 in 2 provided consent to be contacted for further assessment, and 1 in 2 of these proved eligible. We obtained two different samples, which had been collected during the course of another ongoing project. During this time, our imaging studies shifted from a Siemens 1.5 T scanner to a GE 3.0 T scanner. Sample 1 (1.5 T Siemens) consisted of 29 healthy right-handed subjects (17 female) with an average age of 18.2+/−0.62 years with an average education of 12.6+/−0.60 years. Sample 2 (3.0 T GE) consisted of 26 healthy right-handed subjects (24 female) with an average age of 21.0+/−2.6 years with an average education of 14.5+/−1.4 years. All subjects underwent the Structured Clinical Interview for DSM-IV, to identify anxiety and mood disorders and were excluded if they were currently seeking, or had ever sought, treatment for their anxiety or mood symptoms. None of the subjects had taken any psychotropic medications in the prior 12 months. Subjects consumed less than 400 mg of caffeine daily. This study was approved by the SDSU and UCSD Institutional Review Boards and all subjects provided written informed consent to participate All subjects gave their informed, written consent to participate and perform the emotion face processing task during functional magnetic resonance imaging (fMRI).

### Phenotypic Assessments:

A. Behavioral temperament assessment: As described previously¹³, children underwent laboratory-based temperament assessments at one of three ages—21 months, 4 years or 6 years—using age-specific measurement protocols. In brief, the evaluation consisted of behavioral protocols designed to assess the child's reaction to unfamiliar persons and events over a 90-minute battery. In these protocols, the child, with the mother present, encountered a variety of unfamiliar procedures and tasks, including physiological measurements and cognitively challenging tasks administered by an unfamiliar female examiner, and their behavioral responses were observed and quantified. The assessments were videotaped and scored by raters who were blind to the assessment of psychopathology in the children and their parents and blind to genotype status. The relevant dependent variables were behavioral signs of uncertainty, including fretting and crying, cessation of vocalization or activity, retreat, or withdrawal from an unfamiliar event, and frequency of smiles and spontaneous comments (see Rosenbaum et al. 2000 for full description of coded variables). Studies conducted over the past 20 years have established that these variables differentiate inhibited from uninhibited children between age 1 and age 8. As in our previous genetic studies of BI, children were classified as inhibited if they met at least one of three pre-specified categorical definitions of BI¹⁴; children who met none of these definitions were considered unaffected. The genotyped families included 73 children with BI and 89 children without BI (total=162 children). The sample included 77 two-parent families comprising 114 trios, 41 single-parent families (including 17 with more than one genotyped offspring and 6 with phenotyped sibling pairs), and one sibling pair with no parental genotypes.
B. Self-report personality assessment: The NEO-Personality Inventory-Revised (NEO-PI-R) is a widely used, 240-item (plus three validity items) self-report measure of personality, grouped into five major domains: neuroticism (N), extraversion (E), openness to experience (O), conscientiousness (C), and agreeableness (A)¹⁵. Some subjects completed a shorter (60-item) version of the NEO, the NEO-Five Factor Inventory¹⁵, which provides domain scores (expressed as T scores) that are highly correlated with those obtained from the full instrument. T scores were calculated directly from college-age, sex-specific norms. The phenotype of interest was extraversion T scores.
C. fMRI:

Task: During fMRI, each subject was tested on a slightly modified¹⁶ version of the emotion face assessment task (see Hariri et al.)¹⁷. During each 5 second trial, a subject is presented with a target face (on the top of the computer screen) and two probe faces (on the bottom of the screen) and is instructed to match the probe with the same emotional expression to the target by pressing the left or right key on a button box. A block consists of six consecutive trials where the target face is angry, fearful, or happy. During the sensorimotor control task subjects were presented with 5-second trials of either wide or tall ovals or circles in an analogous configuration and instructed to match the shape of the probe to the target. We did not use neutral faces as a comparator condition because there is mounting evidence that neutral faces are not actually processed as neutral¹⁸. Each block of faces and of the sensorimotor control task was presented three times in a pseudo-randomized order. A fixation cross lasting 8 seconds was interspersed between each block presented at the beginning and end of the task (resulting in 14 fixation periods). For each trial, response accuracy and reaction time data were obtained. There were 18 trials (3 blocks of 6 trials) for each face set as well as for shapes. The whole task lasted 512 seconds (matching the scan length).

Image Acquisition (1.5 T): During the task, one BOLD-fMRI run was collected for each subject using a 1.5-Tesla Siemens (Erlangen, Germany) scanner (T2*-weighted echo planar imaging, TR=2000 ms, TE=40 ms, 64×64 matrix, 20 4-mm axial slices, 256 repetitions). During the same experimental session, a T1-weighted image (MPRAGE, TR=11.4 ms, TE=4.4 ms, flip angle=10°, FOV=256×256, 1 mm³ voxels) was obtained for anatomical reference. For preprocessing, voxel time series were interpolated to correct for non-simultaneous slice acquisition within each volume and corrected for three-dimensional motion.

Image Acquisition (3 T): During the task an fMRI run sensitive to blood oxygenation level-dependent (BOLD) contrast was collected for each subject using a Signa EXCITE (GE Healthcare, Milwaukee) 3.0 T scanner (T2* weighted echo planar imaging, TR=2000 ms, TE=32 ms, FOV=250×250 mm3, 64×64 matrix, 30 2.6 mm axial slices with a 1.4 mm gap, 290 scans). fMRI acquisitions were time-locked to the onset of each trial. During the same experimental session, a high resolution T1-weighted image (SPGR, TI=450, TR=8 ms, TE=4 ms, flip angle=12°, FOV=256×256, 1 mm3 voxels) was obtained for anatomical reference.

### Genetic Methods

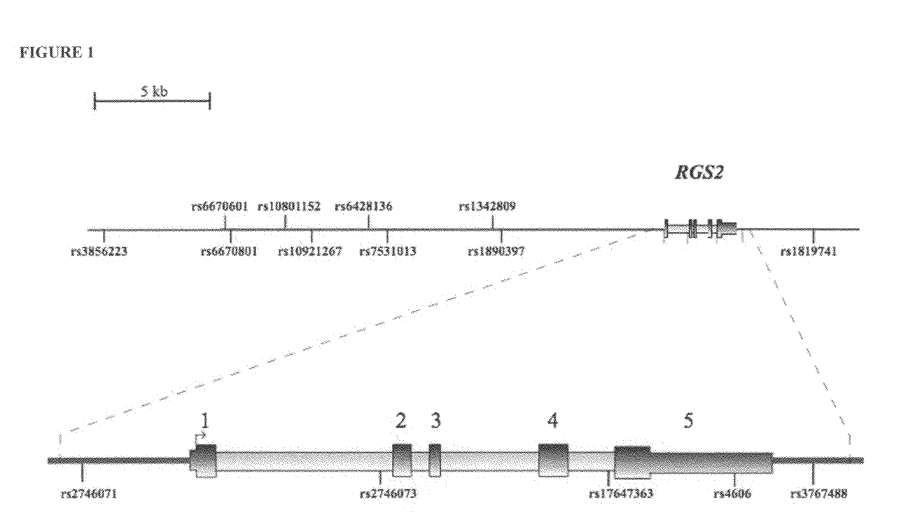

Selection of SNPs: For the analysis of BI temperament, SNPs were selected to capture genetic variation across the RGS2 locus. SNPs were selected from a genomic region comprising the RGS2 gene, 25 kb of 5′ flanking sequence and 10 kb of 3′ flanking sequence as defined in genome build hg 17 using the Phase I International HapMap website (http://www.hapmap.org). Ten tagging SNPs were selected from this region using Tagger (http://www.broad.mit.edu/mpg/tagger/) with r²≧0.8 and the “aggressive” tagging algorithm. We also included five SNPs in the gene (rs2746071, rs2746073, rs17647363, rs4606, rs3767488) that were not available in HapMap at the time of selection but were found in dbSNP (http://www.ncbi.nlm.nih.gov/SNP). Linkage disequilibrium relationships within the SNP set were calculated using the Gabriel criteria as implemented in Haploview (http://www.broad.mit.edu/mpg/haploview/index.php). A single haplotype block encompassed the gene and all markers with the exception of the 5′ marker rs3856223, which nevertheless was in strong linkage disequilibrium (LD) with markers in the block and was thus included in haplotype analysis (Supplementary FIG. 1). The final set of 15 SNPs achieved an average density of 1 SNP/2 kb across a 32 kb region and an average r² of 0.90 with untyped HapMap markers that have minor allele frequency ≧10%, indicating excellent coverage of variation across the locus. Because our marker set included five SNPs not included in HapMap, this average r² is an underestimate of the true variation captured by our set. For the NEO-E analyses, we selected a subset of the 15 markers based on the results of the BI analysis, and for the fMRI analyses we selected rs4606 because of the recent report of its functional significance¹⁹.

Genotyping Methods Genotyping of SNPs in the BI family sample was performed by mass spectrometry (Sequenom, CA). Markers were retained for analysis if they met the following criteria: 1) no significant deviation from Hardy Weinberg equilibrium (p>0.01); 2) minimum call rate of 85% (average call rate was 96.4%). For one SNP (rs4606), genotypes were repeated and the resulting genotypes combined (overall call rate=98%). All 15 markers were thus retained. Affected and unaffected individuals were spread across genotyping plates to avoid bias due to plate-specific genotyping error.

Genotyping for the NEO-E and fMRI samples: SNPs were genotyped with a fluorogenic 5′ nuclease assay method, i.e., the TaqMan technique, using the ABI PRISM 7900 Sequence Detection System (ABI, Foster City, Calif., USA). All genotypes were assayed in duplicate, and discordant genotypes (which ranged from 0 to 0.3%, depending on the marker) were discarded. All markers were in Hardy-Weinberg equilibrium.

Ancestral Proportion Scores. Subjects' ancestries were estimated using a set of unlinked genetic markers by Bayesian cluster analysis, using STRUCTURE software (http://pritch.bsd.uchicago.edu/software.html). The markers were the set of short tandem repeats selected for ancestry information and described previously²⁰. STRUCTURE implements Bayesian cluster modeling that can infer population genetic patterns without prior information of population origins. The model was specified as “admixture” and “allele frequencies correlated,” with 100,000 burn-in and 100,000 MCMC (Markov chain Monte Carlo) iterations. Analysis with STRUCTURE indicated adequate fit for a 3-class solution which was used in the NEO-E and fMRI analyses.

### Statistical Methods

Family-based Association Analysis of Temperament: Family-based association analyses of the BI sample were performed using the Family Based Association Test (FBAT) Program 1.7.1 (http://www.biostat.harvard.edu/˜fbat/default.html). The offset option of FBAT was used to incorporate all offspring who were phenotyped and genotyped. Haplotype-specific and global haplotype tests were performed using permutation (N=100000 cycles) by the hbat-p option. An offset equal to the sample prevalence was incorporated to permit analysis of all offspring who were phenotyped and genotyped. The min-p test was used to calculate a global haplotype test. This test evaluates the statistical significance of the smallest observed p-value among all the individual haplotypes and estimates a p value by permutation. The odds ratio associated with the risk haplotype was calculated from complete trios using WHAP (http://pngu.mgh.harvard.edu/purcell//whap/).

NEO-E (Introversion) Association: Single marker and haplotype-based analyses of the NEO-E quantitative trait was performed using WHAP, with empirical p values determined by permutation testing. Sample mean and variance were fixed to optimize model stability in single marker and haplotype analyses. Analyses incorporated a three-class population solution derived from STRUCTURE to avoid confounding due to population stratification.

Our analytic strategy was based on sequentially maximizing the prior probability of association and reducing multiple testing by first testing the full set of markers for the BI phenotype, retaining SNPs with the strongest signals for the introversion analysis, and then focusing on the most relevant markers for the fMRI analyses. The fMRI analysis focused on rs4606 in particular because it is the marker that has been associated with gene expression levels. To further support these results and confirm they were not a function of genotyping error, we also examined rs10801152, (the SNP that showed the strongest statistical evidence across the BI and introversion analyses).

fMRI Analysis:

Image Processing and Analysis: All structural and functional image processing was done with the Analysis of Functional Neuroimages software package (AFNI) package.²¹ Echoplanar image intensity (EPI) images were co-registered to the 128^{th} image using a 3D-coregistration algorithm. The time series of the alignments in the (x,y,z) and (roll,pitch,yaw) direction was used to obtain motion regressors for each subject. Because small motion corrections are similar in angle (e.g. roll) and displacement (e.g. x), we used only three motion parameters (roll, pitch, yaw) as nuisance regressors to account for motion artifacts. The four orthogonal regressors of interest were: (1) happy, (2) angry, (3) fearful, (4) circle/oval (i.e., shape) sensorimotor condition. These regressors were convolved with a modified gamma variate function to account for the delay and the dispersion brain response of the BOLD-fMRI signal due to hemodynamics^{22,23}. Additional regressors were used to model residual motion in the roll, pitch, and yaw directions as well as baseline and linear trends. The AFNI program 3dDeconvolve was used to calculate the estimated voxel-wise response amplitude. A Gaussian filter with FWHM 4 mm was applied to the voxel-wise percent signal change data to account for individual variations in the anatomical landmarks.

Data of each subject were normalized to Talairach coordinates. Whole brain analyses were followed by a priori analysis of regions of interest (ROIs) using masks (defined by the Talairach demon atlas)²⁴ in the bilateral amygdala, insula, ventromedial prefrontal cortex (vmPFC) and primary visual cortex. Based on these areas of interest, it was determined via simulations that a voxel-wise a-priori probability of 0.05 would result in a corrected cluster-wise activation probability of 0.05 if a minimum volume of 128 μl and 2 connected voxels (in the amygdala, which is a very small structure) or 512 μl and 8 connected voxels (in all other ROIs) was considered. The areas of interest were superimposed on each individual's voxel-wise percent signal change brain image. Only activations within the areas of interest, which also satisfied the volume and voxel connection criteria were extracted and used for further analysis. The corrected voxel-wise probabilities are: amygdala p<0.012, insular cortex p<0.000069, medial prefrontal cortex p<0.00014, and visual cortex p<0.000070. These corrected voxel probabilities are based on Monte Carlo simulations using AFNI's program AlphaSim using the filtered data and the a-priori defined regions of interest.

To maximize power by increasing the number of individuals homozygous for the putative rs4606 risk (“G”) allele, we pooled the 1.5 T and 3 T datasets rather than considering them separately. Specifically, for these analyses we used a regression approach with magnet type (1.5 T or 3 T) and ancestral informative marker (AIM 1 and AIM 2) coefficients as covariates and genotype for rs4606 (CC=0, CG=1, GG=2) as the variable of interest. Areas with a significant gene effect, i.e. voxel-wise partial correlation coefficient with p<0.05, that also fulfilled the volume-threshold cluster condition within the regions of interest were extracted and additional statistical analyses were conducted using SPSS 15.0. Results of the regression analyses are presented in Supplementary Table 1.

### Results

Association of RGS2 with Childhood Anxious Temperament

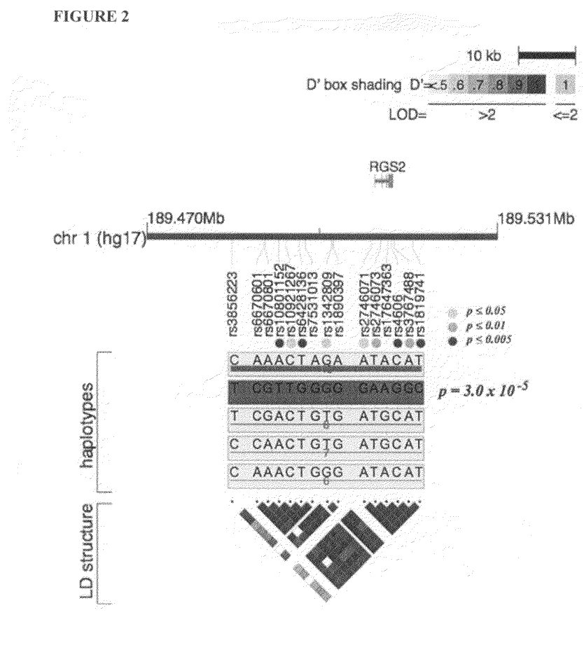

We first examined whether RGS2 influences behavioral and neurobiological phenotypes underlying human anxiety by examining a form of human anxious temperament that shares core phenotypic and biologic features with mouse models of unconditioned and novelty-induced fear behavior. Behavioral inhibition to the unfamiliar (BI) is a heritable temperamental profile characterized by a tendency to be shy, avoidant, and behaviorally-restrained in situations that are novel or unfamiliar²⁵. Biological features of BI include evidence of increased sympathetic tone and limbic hyper-reactivity to novel stimuli^{9,25}. BI is a familial and developmental risk factor for anxiety disorders (and, in particular, SAD)²⁶ but has greater estimated heritability than the diagnostic categories²⁷. The sample comprised 119 families in which children underwent standardized laboratory-based behavioral assessments of BI, as previously described¹³. To capture genetic variation across the RGS2 locus, we genotyped a set of 15 single nucleotide polymorphisms (SNPs) with an average density of 1 SNP/2 kb across a 32 kb region spanning RGS2 (FIG. 1). As shown in Table 1 and FIG. 2, nine of the 15 SNPs tested were associated with BI, including the G allele of the 3′UTR SNP rs4606 (p=0.0026) which has been shown to be associated with reduced RGS2 expression in vitro¹⁹. A haplotype comprising all 15 markers was also associated with BI (permuted p=3.0×10⁻⁵). The odds ratio for BI associated with the risk haplotype, calculated from complete family trios, was 2.99 (95% CI: 1.31-6.84).

TABLE 1
Association of BI with markers spanning
the RGS2 locus
1A. Single marker results

| | Position | | | Z | |
|---|---|---|---|---|---|
| Marker | (hg17) | Alleles* | MAF | (allele)^{#} | p value |
| rs3856223 | 189484723 | C/T | .35 | 1.90 (T) | 0.057 |
| rs6670601 | 189490272 | A/C | .47 | 0.54 (C) | 0.59 |
| rs6670801 | 189490498 | A/G | .36 | 1.51 (G) | 0.13 |
| rs10801152 | 189492961 | A/T | .31 | 2.99 (T) | 0.0028 |
| rs10921267 | 189494228 | C/T | .27 | 2.43 (T) | 0.015 |
| rs6428136 | 189496545 | T/G | .27 | 2.92 (G) | 0.0036 |
| rs7531013 | 189497600 | G/A | .47 | 0.26 (G) | 0.79 |
| rs1342809 | 189502209 | G/T | .17 | 2.05 (G) | 0.040 |
| rs1890397 | 189502590 | G/A | .49 | 0.04 (A) | 0.97 |
| rs2746071 | 189509221 | A/G | .29 | 1.98 (G) | 0.047 |
| rs2746073 | 189510884 | T/A | .25 | 2.57 (A) | 0.010 |
| rs17647363 | 189512155 | A/G | .16 | 1.53 (A) | 0.13 |
| rs4606 | 189512829 | C/G | .27 | 3.01 (G) | 0.0026 |
| rs3767488 | 189513296 | A/G | .25 | 2.78 (G) | 0.0055 |
| rs1819741 | 189516495 | T/C | .26 | 3.07 (C) | 0.0021 |

1B. Haplotype results (>5% frequency) with
permuted p values

| Haplotype | Frequency | p value |
|---|---|---|
| C-A-A-A-C-T-A-G-A-A-T-A-C-A-T | 0.48 | 0.68 |
| T-C-G-T-T-G-G-G-G-G-A-A-G-G-C | 0.18 | 0.000020 |
| C-C-A-A-C-T-G-T-G-A-T-G-C-A-T | 0.08 | 0.75 |
| C-A-A-A-C-T-G-G-G-A-T-A-C-A-T | 0.07 | 0.59 |
| T-C-G-A-C-T-G-T-G-A-T-G-C-A-T | 0.07 | 0.11 |

| | |
|---|---|
| Global minimal p permutation test | 0.000030 |

*major/minor;
MAF = minor allele frequency;
^{#}Z statistic and overtransmitted allele.
Bolded p values are significant after Bonferroni correction for 15 single marker tests.

Association of RGS2 with Social Anxiety-Related Personality in Adults

In adults, social inhibition can be indexed by the personality trait of introversion (low extraversion), which, like BI, is a heritable trait²⁸ characterized by low levels of sociability and aversion to large groups. Longitudinal data suggest that childhood BI is a developmental precursor of introversion (but not neuroticism)²⁹. Like BI, introversion is associated with risk for anxiety disorders including SAD³⁰. If variants in RGS2 are associated with temperamental shyness, we hypothesized that these variants would also be associated with introversion (low extraversion). We genotyped the four markers that showed the strongest signal in the BI sample in an independent sample of 744 college undergraduates (228 male, 516 female) who completed the NEO PI-R, from which the extraversion scale (NEO-E) can be derived¹⁵. Consistent with our prediction, we observed association between NEO-E and the same alleles of these four markers that were associated with BI (Table 2). A haplotype of these four alleles was also associated with introversion (global and haplotype specific p=0.038). Although our primary hypothesis was that RGS2 would be associated with introversion, we performed secondary analyses to determine whether the effect was specific to this trait. In those analyses, we observed no association between RGS2 markers and the other NEO subscales (neuroticism, openness, conscientiousness, or agreeableness).

TABLE 2
Single marker and haplotype association of four RGS2 markers with
introversion.

| | Risk | | |
|---|---|---|---|
| Marker | Alleles* | LRT# | p value |
| rs10801152 | T | 7.18 | 0.0074 |
| rs6428136 | G | 5.54 | 0.019 |
| rs4606 | G | 4.11 | 0.043 |
| rs1819741 | C | 3.81 | 0.051 |
| 4-marker Haplotype† | T-G-G-C | 4.32 | 0.038 |

*allele associated with introversion;
#LRT: likelihood ratio test statistic, 1 df;
†haplotype-specific test, minimum haplotype frequency 5%. Analysis of ancestry informative markers indicated no effect of population stratification.

Association of RGS2 with Social Anxiety-Related Brain Function

In light of previous studies suggesting that BI and social anxiety are mediated by hyperreactivity of brain structures (especially amygdala and insula) thought to underlie anxiety proneness⁹⁻¹², we hypothesized that RGS2 variants associated with BI and introversion would also show association with functional reactivity of these structures during emotion processing. To investigate this, we examined genotype effects on limbic brain reactivity to emotional faces, a neuroimaging assay of anxious temperament. Functional magnetic resonance imaging (fMRI) studies have shown that limbic brain circuits involved in anxiety are activated when individuals view novel or emotional faces³¹. In particular, increased amygdala activation to novel or emotional faces has been associated with inhibited temperament⁹, social anxiety traits³², and social anxiety disorder¹², though other areas, including anterior cingulate cortex and insular cortex, have also been implicated^{31,33}. By directly indexing brain function, anxiety-related fMRI phenotypes may provide more proximal and therefore more powerful measures of gene action.

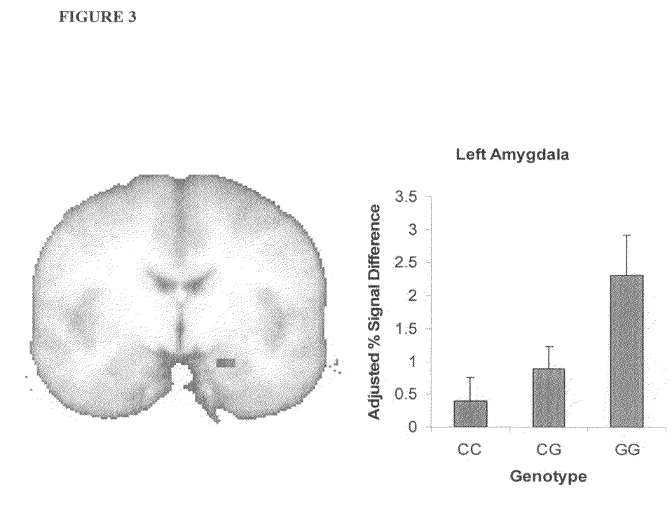

For these analyses, we selected the 3′ UTR SNP rs4606, which has been associated with variation in RGS2 mRNA expression 19 We genotyped rs4606 in two independent groups (n=29 tested in a 1.5 T magnet and n=26 tested in a 3 T magnet) of healthy volunteers drawn from an ongoing study with college-age individuals. To maximize power for analyses of the putative risk allele (rs4606-G), we pooled the samples and included a covariate for the magnets. Subjects in both magnets were tested during fMRI using the same version of a slightly modified emotion face assessment task that has been shown to be sensitive to genetic influence¹⁷. For each 5 second trial, a subject is presented with a target face (on the top of the computer screen) and two probe faces (on the bottom of the screen) and is instructed to match the probe with the same emotional expression (happy, sad, angry) to the target by pressing the left or right key on a button box. During the sensorimotor control task subjects were presented with 5-second trials of either wide or tall ovals or circles in an analogous configuration and instructed to match the shape of the probe to the target. Several investigators have used this task to show significant activations in the amygdala during the presentation of faces versus the sensorimotor control condition^{17,34}. Moreover, we have found previously that the degree of insula activation during this task was modulated by both acute administration of an anxiolytic and by the degree of anxiety proneness^{10,16} The rs4606 G allele, which showed association with BI and introversion in the analyses described above, was significantly associated with the degree of left amygdala and bilateral insular cortex activation (FIGS. 2 and 3 and Table 3).

TABLE 3
Multiple regression analyses of rs4606 genotype for the constrained
regions of interest in the amygdala and left and right insular cortex.
TESLA indicates the 1.5T versus 3T magnet as a covariate and AIM1
and AIM2 refer to ancestry informative marker covariates derived from
STRUCTURE. As shown, the association with rs4606 genotype
explains a significant proportion of the variance in activation,
even after adjusting for these covariates.

| | Tesla | AIM1 | AIM2 | rs4606 |
|---|---|---|---|---|
| Left Insula (1) | | | | |
| Coefficient | 0.17 | −0.75 | −1.29 | 0.74 |
| t | 0.68 | −1.32 | −1.80 | 2.73 |
| p | 0.498 | 0.193 | 0.078 | 0.009 |
| Left Insula (2) | | | | |
| Coefficient | −0.10 | 0.01 | −0.62 | 1.14 |
| t | −0.27 | 0.01 | −0.58 | 2.84 |
| p | 0.790 | 0.993 | 0.563 | 0.007 |
| Right Insula | | | | |
| Coefficient | 0.39 | 0.41 | −0.41 | 0.78 |
| t | 1.64 | 0.74 | −0.60 | 2.97 |
| p | 0.107 | 0.464 | 0.553 | 0.005 |
| Left Amygdala | | | | |
| Coefficient | 0.71 | −1.62 | −2.26 | 0.84 |
| t | 2.28 | −2.22 | −2.46 | 2.40 |
| p | 0.027 | 0.031 | 0.017 | 0.020 |

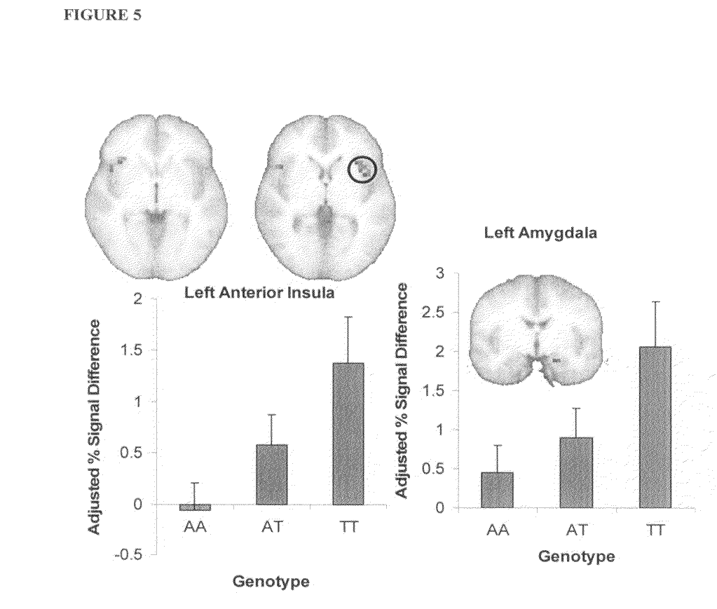

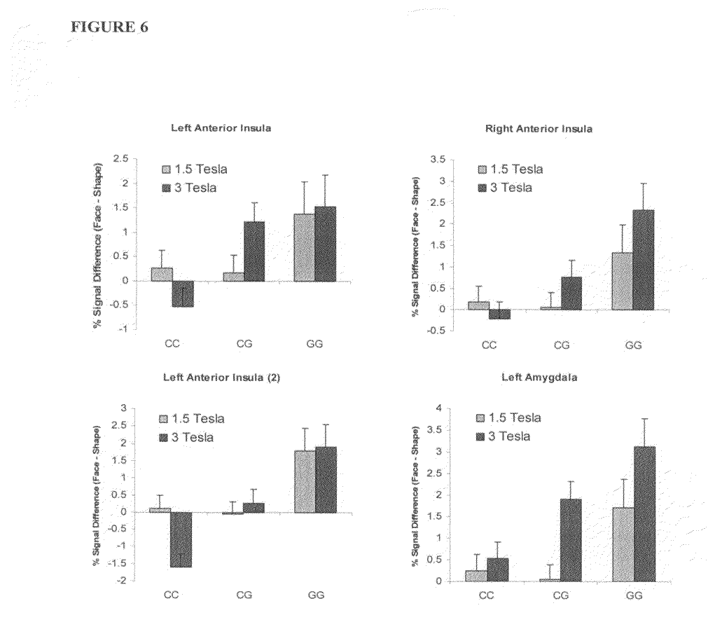

Specifically, in models controlling for magnet (1.5 vs. 3 T) and ancestry informative marker clusters²⁰, the rs4606 G allele was independently associated with the extracted average activation of two clusters in left insula (p=0.009 and p=0.007, respectively), and a cluster in right insula (p=0.005) and left amygdala (p=0.02). The rs4606 SNP accounted for approximately 15% of the variance in amygdala activation and approximately 10-15% of the variance in the insular cortex activation. To corroborate the finding with rs4606, we also examined rs1081152 which in our analyses showed a strong association with both inhibited temperament and introversion. As with rs4606, we observed significant association of the rs1081152 risk allele (T) with left insula (p=0.009) and amygdala (p=0.031) activation (see FIG. 5 and Table 3). Based on the combined volume and voxel-wise p value threshold we did not find any other clusters in the brain associated with the RGS2 SNPs. Finally, in a secondary analysis, we confirmed that the rs4606 genotype effect on insula-amygdala activation was similar in each magnet (1.5 T and 3 T) considered separately (see FIG. 6).

Additional Analyses with rs10801152 SNP:

In order to determine whether the functional regions of interest defined by the conjunction of the anatomical constraint of the amygdala or insula cortex and the volume-thresholded voxel-wise significant effect of rs4606 genotype were also evident with the rs10801152 A/T polymorphism, we used these regions to separately analyze the effect of rs10801152 genotype. (Table 4 and FIG. 5).

TABLE 4
Multiple regression analyses of rs10801152 genotype for the
constrained regions of interest in the amygdala and left and
right insular cortex defined by the rs4606 results. The
rs10801152 T allele (which was also associated with BI and
introversion), was associated with left insula and amygdala
activation. TESLA indicates the 1.5T versus 3T magnet as a
covariate and AIM1 and AIM2 refer to population covariates
derived from STRUCTURE.

| | TESLA | AIM1 | AIM2 | rs10801152 |
|---|---|---|---|---|
| Left | | | | |
| Insula (1) | | | | |
| Coefficient | 0.186 | −0.664 | −1.211 | 0.705 |
| t | 0.781 | −1.184 | −1.720 | 2.735 |
| p | 0.439 | 0.242 | 0.092 | 0.009 |
| Left | | | | |
| Insula (2) | | | | |
| Coefficient | −0.126 | 0.084 | −0.135 | 0.628 |
| t | −0.338 | 0.096 | −0.123 | 1.561 |
| p | 0.737 | 0.924 | 0.903 | 0.125 |
| Right Insula | | | | |
| Coefficient | 0.367 | 0.470 | −0.020 | 0.355 |
| t | 1.493 | 0.812 | −0.027 | 1.337 |
| p | 0.142 | 0.420 | 0.978 | 0.187 |
| Left | | | | |
| Amygdala | | | | |
| Coefficient | 0.721 | −1.544 | −2.130 | 0.735 |
| t | 2.344 | −2.137 | −2.348 | 2.214 |
| p | 0.023 | 0.037 | 0.023 | 0.031 |

### DISCUSSION

Based on consistent results derived from a set of different but interrelated anxiety paradigms in independent samples, we observed compelling evidence that RGS2, the ortholog of a mouse anxiety quantitative trait gene, is also associated with anxiety-related phenotypes in humans. A particularly strong effect was seen for childhood BI which closely parallels behavioral and biologic features of mouse phenotypes influenced by murine Rgs2. Our findings are the first to document association of a specific gene with social anxiety across three levels of phenotypic analysis: a laboratory-based behavioral measure of childhood temperament, a self-report measure of adult personality, and a neuroimaging measure of functional brain activity.

The finding of RGS2-related activation in the amygdala is analogous to similar findings with the serotonin transporter promoter polymorphism¹⁷ and the catechol O-methyltransferase val-met variant³⁴. In addition, our results are the first to demonstrate association between the insula, a limbic brain region involved in emotional processing³³, and a gene implicated in anxiety. The insular cortex is part of a neural system involved in homeostatic processing of autonomic arousal and visceral changes, signaling executive areas to initiate avoidant behavior and altering self-awareness³³. The insular cortex, medial prefrontal cortex, and amygdala play crucial roles in linking internal physiological states to external cues or events. Although some investigators have proposed that the connectivity between amygdala and medial prefrontal cortex or anterior cingulate is a critical genetically determined factor, dysfunction of which predisposes to anxiety or depression³⁵, the limited number of high-risk allele individuals in our sample prevented a rigorous test of this hypothesis using functional connectivity measures. Clearly, future investigation will need to examine genetic determinants of functional connectivity between amygdala or insula and other areas that are important for emotion regulation³⁶.

RGS2 is one of a family of regulators of G protein signaling that function as GTPase accelerating proteins (GAPs), terminating G protein signaling by binding to activated Gα subunits and accelerating the rate of GTP hydrolysis⁶. RGS2 regulates G_{i/o} and Gqα and is expressed in brain regions thought to underlie anxiety including hippocampus, amygdala, cerebral cortex, hypothalamus and dorsal raphe nuclei^{6,37,38}. Neurotransmitters implicated in anxiety, including serotonin, norepinephrine, and dopamine, act at GPCRs. RGS2 has been shown to markedly decrease Gqα signaling by 5HT2A receptors³⁹, which, in turn, play a key role in anxiety and stress responses, as well as response to serotonergic antidepressants. RGS2 has also been shown to regulate hippocampal synaptic plasticity by increasing neurotransmitter release via presynaptic G_{i/o}-mediated Ca²⁺ channel inhibition⁴⁰. Neuronal RGS2 transcription is modulated by plasticity-inducing synaptic stimuli and by agents known to affect anxiety and mood symptoms^{6,38} and RGS2 expression has been implicated in experience-dependent development of neural circuits³⁷. Rgs2-deficient mice exhibit increased anxiety/fear behavior⁴¹, increased sympathetic tone, reduced heart rate variability, altered blood pressure response to a novel environment, and increased urinary norepinephrine excretion⁴²—features also reported in human BI. Our results suggest that at least some genetic influences on fear responses to novelty are evolutionarily conserved. The identity of the specific phenotype-influencing variant(s) mediating RGS2 effects on human anxiety phenotypes cannot be determined from these data, although the dense map of SNPs examined in the analysis of BI capture a minimum of 90% of the genetic variation in the region and is likely to have directly or indirectly assayed the relevant variants. Resequencing of the gene in previous studies has not revealed common coding sequence variants^{19,43} However, the G allele of rs4606, which was associated with anxiety phenotypes in our study, has been associated with reduced RGS2 expression in both peripheral blood mononuclear cells and fibroblasts in hypertensive subjects¹⁹. Reduced RGS2 expression is expected to be associated with anxiety given that deletion of the gene is associated with anxious temperament in mice^{5,41}.

Taken together, our results suggest a model in which genetic variation associated with reduced expression of RGS2 contributes to increased reactivity of limbic brain structures modulating anxious temperament and social anxiety. At a behavioral level, this genetic effect is most evident in direct measurements of inhibited temperament (which itself has been shown to be associated with amygdala reactivity in previous research⁹), with a weaker effect detectable on adult social anxiety-related personality. This model rests in part on the premise that our measures of temperament, personality, and brain function are phenotypically convergent. One way to verify this would be to measure all three phenotypes in the same individuals and examine their relationship; this was not possible because BI is based on laboratory-based behavioral temperament observations in young children (who could not complete the NEO or undergo functional imaging). However, there is substantial evidence that BI, introversion, and limbic reactivity are, in fact, convergent phenotypes: prior studies have demonstrated an association between BI measured and introversion^{29,44}, between BI and limbic reactivity to emotional faces^{9,45}, and between all three traits (BI, introversion, limbic reactivity to emotional faces) and social anxiety disorder^{11,12,30,46-51}.

In secondary analyses examining the specificity of RGS2 effects on personality, we did not observe association with neuroticism which has been reported to be linked to the 1q region syntenic with mouse chromosome 1⁵². Our data do not support the hypothesis that RGS2 underlies this linkage signal as our adult sample was powered to detect loci explaining as little as 1.5% of the variance in neuroticism. However, it should be noted that the linked region contains many genes, and it may be that one or more of these genes contribute to neuroticism. Of note, however, a recent whole genome association study of neuroticism failed to detect any loci at this region⁵³. To our knowledge, no linkage or association studies that include loci on 1q have examined the phenotype of introversion. Prior studies suggest that introversion is more specifically related to BI and social anxiety^{44,50,54,55}, while neuroticism appears to be a non-specific risk factor for depression and anxiety disorders (especially generalized anxiety disorder)^{56,57}. Whereas neuroticism mainly captures negative emotionality and worry, introversion is more directly related to social inhibition and shyness, core features of BI. Caspi and colleagues²⁹ examined temperament in 3 year-old children and followed them to adulthood. Inhibited temperament at age 3 was associated with introversion at age 26 but was unrelated to adult neuroticism. Gladstone and Parker⁴⁴ found that an adult measure of BI was strongly and similarly correlated with introversion (r=0.75) and social anxiety (r=0.77). In a subsequent study, retrospective childhood BI was significantly associated with social phobia but not panic disorder, GAD or agoraphobia⁴⁷ Numerous other studies, including longitudinal studies, have confirmed the specific relationship between BI and social anxiety^{26,48,49} and we have previously shown in a college sample that introversion is highly and significantly correlated with measures of shyness and social anxiety⁵⁸

Further studies will be needed to determine which, if any, anxiety disorder phenotypes are most tightly related to RGS2. Given the association with BI and introversion, two traits that are risk factors for SAD, we would predict that SAD is the most likely anxiety disorder to be associated with RGS2. A recent study by Leygraf and colleagues reported nominally significant evidence of association between RGS2 markers and panic disorder⁵⁹, though these results would not survive correction for multiple testing. To the extent that genetic effects on DSM-IV anxiety disorders may be smaller than effects on the intermediate phenotypes examined here, much larger samples may be needed for studies of the clinical disorders.

In conjunction with studies in mouse models, our findings suggest that RGS2 modulators could provide a novel therapeutic approach for the treatment of anxiety disorders. For example, agents that facilitate RGS2 would be expected to inhibit GPCR signaling in response to neurotransmitters targeted by antidepressants that effectively treat anxiety disorders. The hypothesis that anxiety-proneness is related to reduced RGS2 expression implies that agents that enhance RGS2 activity would be anxiolytic. The regional expression of RGS2 in limbic and paralimbic brain areas coupled with its selectivity for Gqα-mediated and G_{i/o} signaling might enhance the therapeutic action of GPCR-based treatments of anxiety and mood disorders⁶.

### REFERENCES

1. Smoller J, Tsuang M. Panic and phobic anxiety: defining phenotypes for genetic studies. Am J Psychiatry 1998; 155:1152-1162.
2. Kaabi B, Gelemter J, Woods S W, Goddard A, Page G P, Elston R C. Genome scan for loci predisposing to anxiety disorders using a novel multivariate approach: strong evidence for a chromosome 4 risk locus. Am J Hum Genet 2006; 78:543-53.
3. Smoller J W, Aciemo J S, Jr., Rosenbaum J F et al. Targeted genome screen of panic disorder and anxiety disorder proneness using homology to murine QTL regions. Am J Med Genet 2001; 105:195-206.
4. Flint J. Analysis of quantitative trait loci that influence animal behavior. J Neurobiol 2003; 54:46-77.
5. Yalcin B, Willis-Owen S A, Fullerton J et al. Genetic dissection of a behavioral quantitative trait locus shows that Rgs2 modulates anxiety in mice. Nat Genet 2004; 36:1197-202.
6. Neubig R R, Siderovski D P. Regulators of G-protein signalling as new central nervous system drug targets. Nat Rev Drug Discov 2002; 1:187-97.
7. Kagan J. Galen's prophecy. New York: BasicBooks; 1994.
8. Flint J, Corley R, DeFries J C et al. A simple genetic basis for a complex psychological trait in laboratory mice. Science 1995; 269:1432-1435.
9. Schwartz C E, Wright C I, Shin L M, Kagan J, Rauch S L. Inhibited and uninhibited infants “grown up”: adult amygdalar response to novelty. Science 2003; 300:1952-3.
10. Stein M B, Simmons A N, Feinstein J S, Paulus M P. Increased amygdala and insula activation during emotion processing in anxiety-prone subjects. Am J Psychiatry 2007; 164:318-27.
11. Phan K L, Fitzgerald D A, Nathan P J, Tancer M E. Association between amygdala hyperactivity to harsh faces and severity of social anxiety in generalized social phobia. Biol Psychiatry 2006; 59:424-9.
12. Stein M B, Goldin P R, Sareen J, Zorrilla L T, Brown G G. Increased amygdala activation to angry and contemptuous faces in generalized social phobia. Arch Gen Psychiatry 2002; 59:1027-34.
13. Rosenbaum J F, Biederman J, Hirshfeld-Becker D R et al. A controlled study of behavioral inhibition in children of parents with panic disorder and depression. Am J Psychiatry 2000; 157:2002-10.
14. Smoller J, Rosenbaum J, Biederman J et al. Association of a genetic marker at the corticotropin releasing hormone locus with behavioral inhibition. Biol Psychiatry 2003; 54:1376-1381.
15. Costa P T, McCrae R R. NEO-PI-R Professional Manual. Odessa, Fla.: Psychological Assessment Resources; 1992.
16. Paulus M P, Feinstein J S, Castillo G, Simmons A N, Stein M B. Dose-dependent decrease of activation in bilateral amygdala and insula by lorazepam during emotion processing. Arch Gen Psychiatry 2005; 62:282-8.
17. Hariri A R, Mattay V S, Tessitore A et al. Serotonin transporter genetic variation and the response of the human amygdala. Science 2002; 297:400-3.
18. Cooney R E, Atlas L Y, Joormann J, Eugene F, Gotlib I H. Amygdala activation in the processing of neutral faces in social anxiety disorder: is neutral really neutral? Psychiatry Res 2006; 148:55-9.
19. Semplicini A, Lenzini L, Sartori M et al. Reduced expression of regulator of G-protein signaling 2 (RGS2) in hypertensive patients increases calcium mobilization and ERK1/2 phosphorylation induced by angiotensin II. J Hypertens 2006; 24:1115-24.
20. Yang B Z, Zhao H, Kranzler H R, Gelernter J. Practical population group assignment with selected informative markers: characteristics and properties of Bayesian clustering via STRUCTURE. Genet Epidemiol 2005; 28:302-12.
21. Cox R W. AFNI: software for analysis and visualization of functional magnetic resonance neuroimages. Comput Biomed Res 1996; 29: 162-73.
22. Cohen M S. Parametric analysis of fMRI data using linear systems methods. Neuroimage 1997; 6:93-103.
23. Friston K J, Frith C D, Turner R, Frackowiak R S. Characterizing evoked hemodynamics with fMRI. Neuroimage 1995; 2:157-65.
24. Lancaster J L, Woldorff M G, Parsons L M et al. Automated Talairach atlas labels for functional brain mapping. Hum Brain Mapp 2000; 10:120-31.
25. Kagan J, Reznick S, Snidman N. Biological bases of childhood shyness. Science 1988; 240:167-171.
26. Biederman J, Hirshfeld-Becker D R, Rosenbaum J F et al. Further evidence of association between behavioral inhibition and social anxiety in children. Am J Psychiatry 2001; 158:1673-9.
27. Robinson J, Kagan J, Reznick J, Corley R. The heritability of inhibited and uninhibited behavior: a twin study. Developmental Psychology 1992; 28:1030-1037.
28. Jang K L, Livesley W J, Vernon P A. Heritability of the big five personality dimensions and their facets: a twin study. J Pers 1996; 64:577-91.
29. Caspi A, Harrington H, Milne B, Amell J W, Theodore R F, Moffitt T E. Children's behavioral styles at age 3 are linked to their adult personality traits at age 26. J Pers 2003; 71:495-513.
30. Bienvenu O J, Nestadt G, Samuels J F, Costa P T, Howard W T, Eaton W W. Phobic, panic, and major depressive disorders and the five-factor model of personality. J Nerv Ment Dis 2001; 189:154-61.
31. Phan K L, Wager T D, Taylor S F, Liberzon I. Functional neuroimaging studies of human emotions. CNS Spectr 2004; 9:258-66.
32. Killgore W D, Yurgelun-Todd D A. Social anxiety predicts amygdala activation in adolescents viewing fearful faces. Neuroreport 2005; 16:1671-5.
33. Paulus M P, Stein M B. An insular view of anxiety. Biol Psychiatry 2006; 60:383-7.
34. Drabant E M, Hariri A R, Meyer-Lindenberg A et al. Catechol O-methyltransferase val158met genotype and neural mechanisms related to affective arousal and regulation. Arch Gen Psychiatry 2006; 63:1396-406.
35. Pezawas L, Meyer-Lindenberg A, Drabant E M et al. 5-HTTLPR polymorphism impacts human cingulate-amygdala interactions: a genetic susceptibility mechanism for depression. Nat Neurosci 2005; 8:828-34.
36. Meyer-Lindenberg A, Weinberger D R. Intermediate phenotypes and genetic mechanisms of psychiatric disorders. Nat Rev Neurosci 2006; 7:818-27.
37. Ingi T, Aoki Y. Expression of RGS2, RGS4 and RGS7 in the developing postnatal brain. Eur J Neurosci 2002; 15:929-36.
38. Ingi T, Krumins A M, Chidiac P et al. Dynamic regulation of RGS2 suggests a novel mechanism in G-protein signaling and neuronal plasticity. J Neurosci 1998; 18:7178-88.
39. Ghavami A, Hunt R A, Olsen M A et al. Differential effects of regulator of G protein signaling (RGS) proteins on serotonin 5-HT1A, 5-HT2A, and dopamine D2 receptor-mediated signaling and adenylyl cyclase activity. Cell Signal 2004; 16:711-21.
40. Han J, Mark M D, Li X et al. RGS2 Determines Short-Term Synaptic Plasticity in Hippocampal Neurons by Regulating G(i/o)-Mediated Inhibition of Presynaptic Ca(2+) Channels. Neuron 2006; 51:575-86.
41. Oliveira-Dos-Santos A J, Matsumoto G, Snow B E et al. Regulation of T cell activation, anxiety, and male aggression by RGS2. Proc Natl Acad Sci USA 2000; 97:12272-7.
42. Gross V, Tank J, Obst M et al. Autonomic nervous system and blood pressure regulation in RGS2-deficient mice. Am J Physiol Regul Integr Comp Physiol 2005; 288:R1134-42.
43. Riddle E L, Rana B K, Murthy K K et al. Polymorphisms and haplotypes of the regulator of G protein signaling-2 gene in normotensives and hypertensives. Hypertension 2006; 47:415-20.
44. Gladstone G, Parker G. Measuring a behaviorally inhibited temperament style: development and initial validation of new self-report measures. Psychiatry Res 2005; 135:133-43.
45. Perez-Edgar K, Roberson-Nay R, Hardin M G et al. Attention alters neural responses to evocative faces in behaviorally inhibited adolescents. Neuroimage 2007; 35:1538-46.
46. Hayward C, Killen J, Kraemer H, Taylor C. Linking self-reported childhood behavioral inhibition to adolescent social phobia. J Am Acad Child Adolesc Psychiatry 1998; 37:1308-1316.
47. Gladstone G L, Parker G B, Mitchell P B, Wilhelm K A, Malhi G S. Relationship between self-reported childhood behavioral inhibition and lifetime anxiety disorders in a clinical sample. Depress Anxiety 2005; 22:103-13.
48. Hirshfeld-Becker D R, Biederman J, Henin A et al. Behavioral inhibition in preschool children at risk is a specific predictor of middle childhood social anxiety: a five-year follow-up. J Dev Behav Pediatr 2007; 28:225-33.
49. Schwartz C, Snidman N, Kagan J. Adolescent social anxiety as an outcome of inhibited temperament in childhood. J Am Acad Child Adolesc Psychiatry 1999; 38:1008-1015.
50. Bienvenu O J, Samuels J F, Costa P T, Reti I M, Eaton W W, Nestadt G. Anxiety and depressive disorders and the five-factor model of personality: a higher- and lower-order personality trait investigation in a community sample. Depress Anxiety 2004; 20:92-7.
51. Lorberbaum J P, Kose S, Johnson M R et al. Neural correlates of speech anticipatory anxiety in generalized social phobia. Neuroreport 2004; 15:2701-5.
52. Fullerton J, Cubin M, Tiwari H et al. Linkage analysis of extremely discordant and concordant sibling pairs identifies quantitative-trait loci that influence variation in the human personality trait neuroticism. Am J Hum Genet 2003; 72:879-90.
53. Shifman S, Bhomra A, Smiley S et al. A whole genome association study of neuroticism using DNA pooling. Mol Psychiatry 2007.
54. Bienvenu O J, Brown C, Samuels J F et al. Normal personality traits and comorbidity among phobic, panic and major depressive disorders. Psychiatry Res 2001; 102:73-85.
55. Hummelen B, Wilberg T, Pedersen G, Karterud S. The relationship between avoidant personality disorder and social phobia. Compr Psychiatry 2007; 48:348-56.
56. Hettema J M, Prescott C A, Kendler K S. Genetic and environmental sources of covariation between generalized anxiety disorder and neuroticism. Am J Psychiatry 2004; 161:1581-7.
57. Kendler K S, Gardner C O, Gatz M, Pedersen N L. The sources of co-morbidity between major depression and generalized anxiety disorder in a Swedish national twin sample. Psychol Med 2007; 37:453-62.
58. Stein M B, Schork N J, Gelernter J. A polymorphism of the beta1-adrenergic receptor is associated with low extraversion. Biol Psychiatry 2004; 56:217-24.
59. Leygraf A, Hohoff C, Freitag C et al. Rgs 2 gene polymorphisms as modulators of anxiety in humans? J Neural Transm 2006.

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A method for diagnosing a genetic predisposition or susceptibility for an anxiety disorder in a human individual comprising:
detecting in a sample obtained from the individual one or more genetic markers in a RGS2 nucleic acid or fragment thereof,
wherein the presence of the one or more markers indicates that the individual has a genetic predisposition or susceptibility for an anxiety disorder.

2. The method of claim 1, wherein the one or more genetic markers are rs3856223, rs6670601, rs6670801, rs10801152, rs10921267, rs6428136, rs7531013, rs1342809, rs1890397, rs2746071, rs2746073, rs17647363, rs4606, rs3767488 and/or rs1819741.

3. The method of claim 2, wherein the one or more markers are rs10801152, rs10921267, rs6428136, rs1342809, rs2746071, rs2746073, rs4606, rs3767488 and/or rs1819741.

4. The method of claim 2, wherein the one or more markers are rs10801152, rs4606 and/or rs1819741.

5. The method of claim 4, wherein the one or more markers is rs4606.

6. The method of claim 1, wherein the method comprises detecting a haplotype comprising the markers rs10801152, rs4606, rs6428136 and rs1819741.

7. The method of claim 1, wherein the method comprises detecting a haplotype comprising all 15 markers.

8. The method of claim 1, wherein the anxiety disorder is inhibited temperament (BI), introversion, panic anxiety disorder, phobic anxiety disorder, or social anxiety disorder (SAD).

9. The method of claim 1, wherein the anxiety disorder is social phobia.

10. The method of claim 1, wherein the nucleic acid is DNA, genomic DNA, RNA, cDNA, hnRNA or mRNA.

11. The method of claim 1, wherein the detection is accomplished by sequencing, hybridization, restriction fragment analysis, oligonucleotide ligation assay or allele specific PCR.

12. A diagnostic kit and/or a research kit, comprising at least one combination of probes for detecting at least one of the markers according to claim 1.

13. A method of treatment or prophylaxis of an anxiety disorder, comprising
performing the diagnostic method according to claim 1 to identify an individual that has a genetic predisposition or susceptibility for an anxiety disorder, and
administering to the individual a therapeutically effective amount of a composition suitable to delay, reduce or prevent the anxiety disorder in the individual and/or treating the individual with therapy.

14. The method of claim 13, wherein the composition comprises an antidepressant compound.

15. The method of claim 14, wherein the antidepressant compound is monoamine oxidase inhibitor (MAOI) such as Harmaline, Iproclozide, Iproniazid, Isocarboxazid, Nialamide, Phenelzine, Selegiline, Toloxatone, Tranylcypromine; a reversible inhibitor of monoamine oxidase A (RIMA) such as Brofaromine and Moclobemide; a dopamine reuptake inhibitor (DARI) such as Amineptine, Phenmetrazine, Vanoxerine and Modafinil; a norepinephrine-dopamine reuptake inhibitor such as Bupropion; a norepinephrine reuptake inhibitor (NRI) or (NARI) such as Atomoxetine, Maprotiline, Reboxetine and Viloxazine; a serotonin-norepinephrine reuptake inhibitor (SNRI) such as Duloxetine, Milnacipran and Venlafaxine; a selective serotonin reuptake inhibitor (SSRI) such as Alaproclate, Etoperidone, Citalopram, Escitalopram, Fluoxetine, Fluvoxamine, Paroxetine, Sertraline and Zimelidine; a selective serotonin reuptake enhancer (SSRE) such as Tianeptine; a tricyclic antidepressant (TCA) such as Amitriptyline, Amoxapine, Butriptyline, Clomipramine, Desipramine, Dibenzepin, Dothiepin, Doxepin, Imipramine, Iprindole, Lofepramine, Melitracen, Nortriptyline, Opipramol, Protriptyline and Trimipramine; a tetracyclic antidepressant such as Maprotiline, Mianserin, Nefazodone and Trazodone; or a noradrenergic and specific serotonergic antidepressant (NaSSA) such as Mirtazapine.

16. The method of claim 13, wherein the composition comprises a RGS2 modulator.

17. The method of claim 16, wherein the RGS2 modulator is a siRNA molecule that reduces RGS2 expression.

18. The method of claim 16, wherein the RGS2 modulator is an expression vector that increases RGS2 expression.

19. The method of claim 13, wherein the composition comprises an antidepressant and a RGS2 modulator.

20. The method of claim 19, wherein the antidepressant and the RGS2 modulator are administered separately.

21. The method of claim 13 wherein the therapy is psychotherapy, cognitive-behavioral therapy and/or behavior therapy.
